(19) United States
(12) Patent Application Publication
(10) Pub. No.: US 2019/0013114 A1
(43) Pub. Date: Jan. 10, 2019

(54) CABLE FOR TRANSMITTING POWER AND METHOD

(71) Applicant: Vincent Rodriguez, Chandler, AZ (US)

(72) Inventor: Vincent Rodriguez, Chandler, AZ (US)

(21) Appl. No.: 16/029,450

(22) Filed: Jul. 6, 2018

Related U.S. Application Data
(60) Provisional application No. 62/529,388, filed on Jul. 6, 2017.

(51) Int. Cl. H01B 7/40; H01B 7/04; H01B 7/17; H01R 31/06

(52) U.S. Cl. CPC H01B 7/40; H01B 7/04; A61M 37/0076; H01R 31/06; H01B 7/17

(57) ABSTRACT

A cable for transmitting power to a tattoo gun from a primary power source is disclosed herein. The cable for transmitting power includes an elongate member having a first end portion and a second end portion, a first adapter located at the first end portion, a second adapter located at the second end portion, and at least one magnet coupled to the second end portion and configured to removably attach the cable to a surface. The cable is useful for delivering power to a tattoo gun while allowing for efficient use and storage.

### CROSS-REFERENCE TO RELATED APPLICATION(S)

The present application is related to and claims priority to U.S. Provisional Patent Application No. 62/529,388 filed Jul. 6, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of tattoo machines of existing art and more specifically relates to tattoo machine accessories.

### RELATED ART

A tattoo machine is a hand-held device generally used to create a tattoo, a permanent marking of the skin with indelible ink. Modern tattoo machines use electromagnetic coils to move an armature bar up and down. Connected to the armature bar is a barred needle grouping that pushes ink into the skin. Tattoo artists generally use the term “machine”, or even “iron”, to refer to their equipment. The word “gun” is often used but many tattoo professionals do not prefer it. In addition to “coiled” tattoo machines there are also Rotary Tattoo Machines, which are powered by regulated motors rather than electromagnetic coils.

To provide power to these machines, tattoo artists typically use a zip/clip cord. This cord can be cross-contaminated and can be difficult to store. People may be inconvenienced by having to clean or throw away a zip/clip cord. An effective alternative is needed.

U.S. Pat. No. 7,442,042 to Justin A. Lewis relates to a magnetic clip cord. The described magnetic clip cord includes a magnetic clip cord for a tattoo machine that comprises a pair of magnet bars, in which one magnet bar is mounted to an electrical cord and the other magnet bar is mounted on the frame of a tattoo machine. The invention consists of an electrical cord, a spring, a set screw, two steel bars, two brass connectors, two non-conductive collars, two magnet bars, and a tattoo machine. The invention is used by aligning the two magnets bar and allowing the magnetic force to connect the two pieces, upon which a positive current will pass along to a tattoo machine when in use. The steel bars carry the negative current and shall connect upon alignment of the magnet bars. The primary feature of the present invention is to prevent tattoo artisan errors attributed to outside forces acting on the power cord that attaches to the tattoo machine being used, by providing a magnetic clip cord design.

### SUMMARY OF THE INVENTION

In view of the foregoing disadvantages inherent in the known tattoo machine accessories art, the present disclosure provides a novel cable for transmitting power and method. The general purpose of the present disclosure, which will be described subsequently in greater detail, is to provide a quick release tattoo gun power cord that may help to prevent cross-contamination while increasing longevity of the cord itself.

A cable for transmitting power to a tattoo gun from a primary power source is disclosed herein. The cable for transmitting power may include an elongate member having a first end portion and a second end portion, a first adapter located at the first end portion, a second adapter located at the second end portion, and at least one magnet coupled to the second end portion and configured to removably attach the cable to a surface. The cable may be useful for delivering power to a tattoo gun while allowing for efficient use and storage.

According to another embodiment, a method of using a cable for transmitting power to a tattoo gun is also disclosed herein. The method of using a cable for transmitting power to a tattoo gun includes providing the cable for transmitting power, attaching the at least one magnet to a toolbox, and attaching the second adapter to the tattoo gun.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a cable for transmitting power and method, constructed and operative according to the teachings of the present disclosure.

FIG. 1 is a perspective view of the cable for transmitting power during an ‘in-use’ condition, according to an embodiment of the disclosure.

FIG. 2 is an alternative perspective view of the cable for transmitting power of FIG. 1, according to an embodiment of the present disclosure.

FIG. 3 is a front view of the cable for transmitting power of FIG. 1, according to an embodiment of the present disclosure.

FIG. 4 is a perspective view of the cable for transmitting power of FIG. 1, according to an embodiment of the present disclosure.

FIG. 5 is a flow diagram illustrating a method of using a cable for transmitting power to a tattoo gun, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to tattoo machine accessories and more particularly to a cable for transmitting power and method as used to provide a quick release tattoo gun power cord that may help to prevent cross-contamination while increasing longevity of the cord itself.

Generally, the present invention provides a magnetic zip tie capable of attaching a tattoo gun cord to a metal toolbox. This device may prevent cross-contamination and extend the longevity of a clip cord. As designed, the device helps to store a clip cord in a more convenient and sanitary manner. This may further allow tattoo artists to connect the cord to a foot pedal, power supply, and tattoo gun, thereby allowing for the pressuring of the foot pedal to release current to a needle bar powering the gun. In a preferred embodiment, the present invention may utilize heat-shrink wrapping to cover all exposed electrical terminals.

Specifically, the present invention may include a magnetic quick release tattoo gun cord and adapters. The cord may allow a user to magnetically store their tattoo gun and power cable cord on a metal tattoo box. The cord may consist of a two-conductor, 16-gauge wire with an “O” terminal on each end connected by a rivet to the needle bar end through a small piece of industrial-strength zip tie. Heat shrink covering can be used to prevent exposure of all electrical terminals. The heat shrink can also cover heavy-duty magnets which may allow the cable to hang onto the metal box. The cord device can be constructed using a 0.25″ mono plug attached to a speaker wire. The cords may measure approximately 6-8′ in length. Exact size, measurement, construction, and design specifications may vary upon manufacturing.

Upon reading this specification, it should be appreciated that, under appropriate circumstances, considering such issues as user preferences, design preference, structural requirements, marketing preferences, cost, available materials, technological advances, etc., other cord arrangements such as, for example, alternative adapters, etc., may be sufficient.

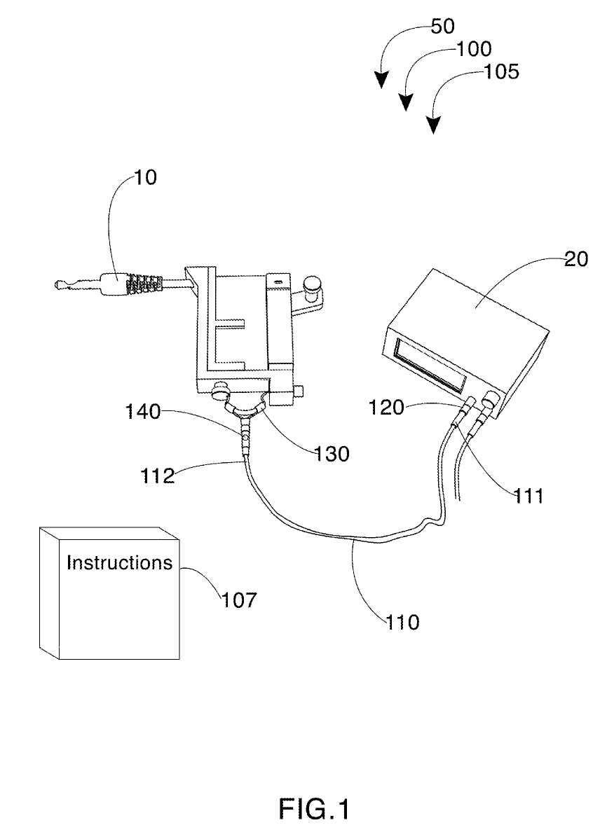

Referring now more specifically to the drawings by numerals of reference, there is shown in FIGS. 1-4, various views of a cable for transmitting power 100.

FIG. 1 shows a cable for transmitting power 100 to a tattoo gun 10 from a primary power source 20 during an ‘in-use’ condition 50, according to an embodiment of the present disclosure. Here, the cable for transmitting power 100 may be beneficial for use by a user to provide a quick release tattoo gun power cord that may help to prevent cross-contamination while increasing longevity of the cord itself. As illustrated, the cable for transmitting power 100 may include an elongate member 110 having a first end portion 111 and a second end portion 112, a first adapter 120, a second adapter 130, and at least one magnet 140 coupled to the second end portion 112 and configured to removably attach the cable 100 to a surface. The first adapter 120 may be located at the first end portion 111 and also be configured to attach to the primary power source 20. The second adapter 130 may be located at the second end portion 112 and configured to attach to the tattoo gun 10.

According to one embodiment, the cable for transmitting power 100 may be arranged as a kit 105. In particular, the cable for transmitting power 100 may further include a set of instructions 107. The instructions 107 may detail functional relationships in relation to the structure of the cable for transmitting power 100 such that the cable for transmitting power 100 can be used, maintained, or the like, in a preferred manner.

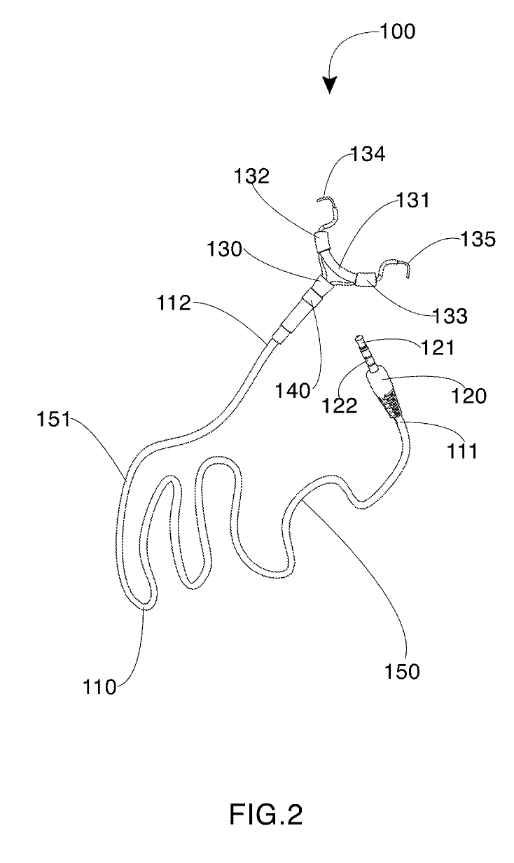

FIG. 2 shows the cable for transmitting power 100 of FIG. 1, according to an embodiment of the present disclosure. As above, the cable for transmitting power 100 may include an elongate member 110 having a first end portion 111 and a second end portion 112, a first adapter 120, a second adapter 130, and at least one magnet 140 coupled to the second end portion 112. The cable 100 may further include a plurality of lumens 150 and have a predetermined length 151 that extends from the first adapter 120 to the second adapter 130. In some versions, cable 100 includes two lumens 150. The first adapter 120 may feature a first portion 121 and a second portion 122, where the second portion 122 may have a diameter greater than a diameter of the first portion 121. The second adapter 130 may comprise a U-shaped member 131 that has a left portion 132 and a right portion 133. The left portion 132 of the U-shaped member 131 may have a left conductor arm 134 for electrical engagement with the tattoo gun 10 (see FIG. 1). Similarly, the right portion 133 of the U-shaped member 131 may have a right conductor arm 135 for electrical engagement with the tattoo gun 10 (see FIG. 1).

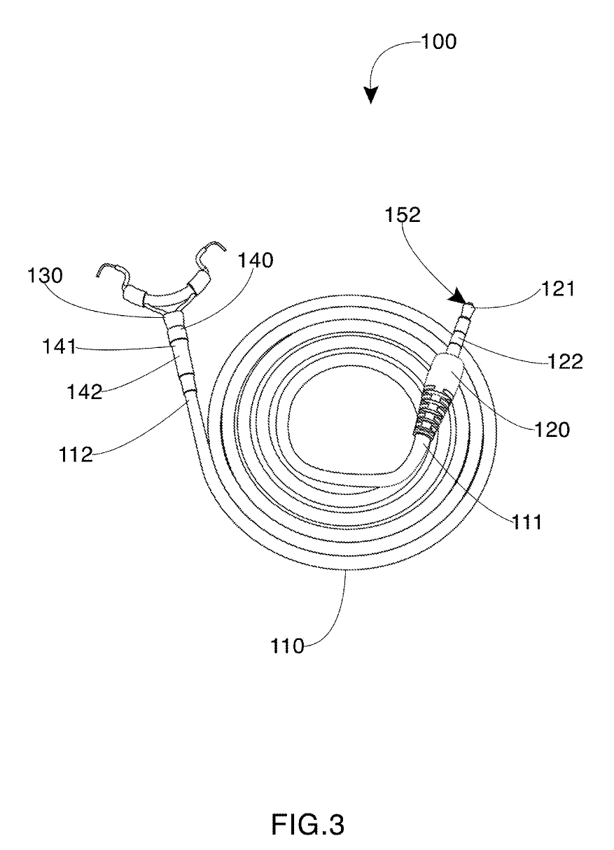

FIG. 3 is a front view of the cable for transmitting power 100 of FIG. 1, according to an embodiment of the present disclosure. Here again, the cable for transmitting power 100 may include an elongate member 110 having a first end portion 111 and a second end portion 112, a first adapter 120, a second adapter 130, and at least one magnet 140 coupled to the second end portion 112. The at least one magnet 140 may be coupled to the second end portion 112 of the elongate member 110 by means of a protective cover 141. This protective cover 141 may include a heat-shrink material 142. Again, the first adapter 120 may feature a first portion 121 and a second portion 122, where the second portion 122 may have a diameter greater than a diameter of the first portion 121. In addition, the first portion 121 may be made of a gold-plated material 152.

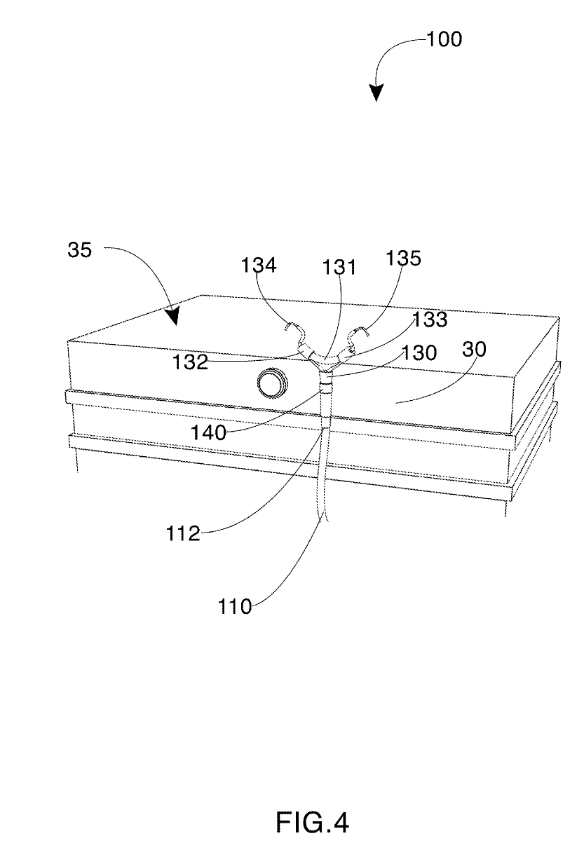

FIG. 4 is a perspective view of the cable for transmitting power 100 of FIG. 1, according to an embodiment of the present disclosure. As above, the cable for transmitting power 100 may include an elongate member 110 having a second end portion 112, a second adapter 130, and at least one magnet 140 coupled to the second end portion 112. The second adapter 130 may comprise a U-shaped member 131 that has a left portion 132 and a right portion 133. The left portion 132 of the U-shaped member 131 may have a left conductor arm 134, while the right portion 133 of the U-shaped member 131 may have a right conductor arm 135. The at least one magnet 140 may be configured to removably attach the cable 100 to a surface 30. In a preferred embodiment, the surface 30 may be a toolbox 35.

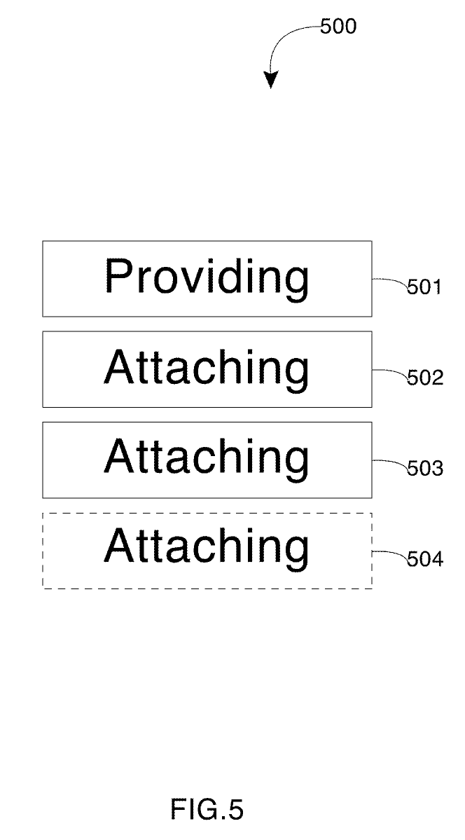

FIG. 5 is a flow diagram illustrating a method of using 500 a cable for transmitting power to a tattoo gun, according to an embodiment of the present disclosure. In particular, the method of using 500 a cable for transmitting power to a tattoo gun may include one or more components or features of the cable for transmitting power 100 as described above. As illustrated, the method of using 500 a cable for transmitting power to a tattoo gun may include the steps of: step one 501, providing an elongate member having a first end portion and a second end portion, a first adapter, the first adapter located at the first end portion of the elongate member, a second adapter, the second adapter located at the second end portion of the elongate member, at least one magnet, and the at least one magnet coupled to the second end portion; step two 502, attaching the at least one magnet to a toolbox; and step three 503, attaching the second adapter to the tattoo gun.

It should be noted that step four 504, attaching the first adapter to a primary power source, is an optional step and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for the cable for transmitting power 100 are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A cable for transmitting power to a tattoo gun from a primary power source, said cable comprising:
an elongate member having a first end portion and a second end portion and configured to transmit power;
a first adapter, said first adapter located at said first end portion and configured to attach to said primary power source;
a second adapter, said second adapter located at said second end portion and configured to attach to said tattoo gun; and
at least one magnet, said at least one magnet coupled along said elongate member and configured to removably attach said cable to a surface.

2. The cable of claim 1, wherein said elongate member is flexible.

3. The cable of claim 2, wherein said elongate member has a plurality of lumens.

4. The cable of claim 3, wherein said plurality of lumens comprises two lumens.

5. The cable of claim 1, wherein said cable has a predetermined length that extends from said first adapter to said second adapter.

6. The cable of claim 5, wherein said predetermined length is approximately six feet.

7. The cable of claim 1, wherein said first adapter has a first portion and a second portion.

8. The cable of claim 7, wherein said second portion has a diameter greater than a diameter of said first portion.

9. The cable of claim 7, wherein said first portion is made of a gold-plated material.

10. The cable of claim 1, wherein said second adapter comprises a U-shaped member.

11. The cable of claim 10, wherein said U-shaped member has a first portion and a second portion.

12. The cable of claim 11, wherein said first portion of said U-shaped member has a first conductor arm for engagement with said tattoo gun.

13. The cable of claim 11, wherein said second portion of said U-shaped member has a second conductor arm for engagement with said tattoo gun.

14. The cable of claim 1, wherein said at least one magnet is coupled along said elongate member by means of a protective cover.

15. The cable of claim 14, wherein said protective cover is a heat-shrink material.

16. The cable of claim 1, wherein said surface is a toolbox.

17. A cable for transmitting power to a tattoo gun from a primary power source, said cable comprising:
an elongate member having a first end portion and a second end portion and configured to transmit power;
a first adapter, said first adapter located at said first end portion and configured to attach to said primary power source;
a second adapter, said second adapter located at said second end portion and configured to attach to said tattoo gun; and
at least one magnet, said at least one magnet coupled to said second end portion and configured to removably attach said cable to a surface;
wherein said elongate member is flexible;
wherein said elongate member has a plurality of lumens;
wherein said plurality of lumens comprises two lumens;
wherein said cable has a predetermined length that extends from said first adapter to said second adapter;
wherein said predetermined length is approximately six feet;
wherein said first adapter has a first portion and a second portion;
wherein said second portion has a diameter greater than a diameter of said first portion;
wherein said first portion is made of a gold-plated material;
wherein said second adapter comprises a U-shaped member;
wherein said U-shaped member has a first portion and a second portion;
wherein said first portion of said U-shaped member has a first conductor arm for engagement with said tattoo gun;
wherein said second portion of said U-shaped member has a second conductor arm for engagement with said tattoo gun;
wherein said at least one magnet is coupled along said elongate member by means of a protective cover;
wherein said protective cover is a heat-shrink material; and
wherein said surface is a toolbox.

18. The cable of claim 17, further comprising set of instructions; and wherein the cable for transmitting power to a tattoo gun from a primary power source and the instructions are arranged as a kit.

19. A method of using a cable for transmitting power to a tattoo gun, the method comprising the steps of:
providing an elongate member having a first end portion and a second end portion,
a first adapter, said first adapter located at said first end portion of said elongate member,
a second adapter, said second adapter located at said second end portion of said elongate member, at least one magnet, said at least one magnet coupled to said second end portion; and
attaching said at least one magnet to a toolbox; and
attaching said second adapter to said tattoo gun.

20. The method of claim 19, further comprising the step of:
attaching said first adapter to a primary power source.
